(19) United States
(12) Patent Application Publication
(10) Pub. No.: US 2019/0151135 A1
(43) Pub. Date: May 23, 2019

(54) Ostomy Support System

(71) Applicant: Sean Swarthout, San Diego, CA (US)

(72) Inventor: Sean Swarthout, San Diego, CA (US)

(21) Appl. No.: 15/820,983

(22) Filed: Nov. 22, 2017

(51) Int. Cl. A61F 5/449

(52) U.S. Cl. CPC A61F 5/449

(57) ABSTRACT

An ostomy support device having a housing extending between a first end and a second end and a base member extending between said first end and said second end. A cavity adapted for protective positioning of a pouch is formed in-between a central portion of an elongated member engaged at both ends to the base member and a facing surface of the base member. One or a plurality of ridges positioned on the elongated member form stiffeners resisting bending of the elongated member. A belt pathway may also be formed between two such ridges positioned on the elongated member.

### BACKGROUND OF THE INVENTION

### 1. Field of the Invention

The present invention relates generally to medical garments and support components. More particularly, it relates to an ostomy belt and support device adapted for waist or ankle positioning by a user, which when in the waist positioning provides a mount and an impact shield for an ostomy bag as well as an undergarment-positioned pathway for a clothing belt such as is used with a pair of pants.

### 2. Prior Art

A colostomy is a surgical procedure which positions an end of the large intestine in a communication through the abdominal wall. This surgical procedure thereby positions a stoma or formed opening in the skin of the patient in the abdomen area, whereafter a pouch is operatively engaged for collecting discharge from the intestine.

A primary problem faced by patients, subsequent to a colostomy, is that of the potential for leakage around the engagement of the collection component which is attached to the stoma location. Conventionally, such collection is provided by a pouch which is positioned to collect and hold bodily drainage. Such pouches can be held by belts or adhesively engaged to the underlying skin surrounding the collection site. As a general rule, relative movement of the pouch and the surrounding skin area and the adhesive material holding it in place, can form a path for leakage. As a consequence, while many patients desire and strive to enjoy a normal and active lifestyle, problems associated with such leakage can cause significant problems.

Patients subsequent to colostomy procedures also are frequently challenged by skin irritation in the area of the pouch where it adheres to the skin. Such can be exacerbated by forces tugging on the pouch and thus the adhesive engagement. Still further, there is a constant worry for patients having such collection pouches, that impacts against the pouch while operatively positioned can cause dismounting or leakage.

The ostomy support system herein described and disclosed provides for a frame forming a surrounding protective support of collection pouches and the like, employed for waste collection. The device features a housing which has an elongated or curved member portion engaged at both ends to a base and to an elastic belt adapted to hold the device in place on the patient. In-between the base and elongated member portion is located a cavity. This cavity, in combination with the elongated member, provides a protective covering and support for an ostomy pouch or bag therein to protect it from impacts while being worn and against contact from a seat belt while riding in a vehicle.

It should be noted, the foregoing examples of related art for ostomy belts and supports and the like and limitations related therewith are intended to be illustrative and not exclusive, and they do not imply any limitations on the ostomy support system described and claimed herein. Various limitations of the related art are already known or will become apparent to those skilled in the art upon a reading and understanding of the specification below and the accompanying drawings.

An object of the present invention is the provision of an ostomy pouch support device, which is securely positionable upon the patient and will both support and protect the collection pouch.

Another object of this invention is the provision of such an ostomy pouch support device which positions a reinforced protective arched beam above the held pouch to shield it from impacts and from compression forces such as a seat belt placed across the front of the user in a vehicle.

Another object of the present invention is to provide such an ostomy collection pouch support device which additionally provides a formed pathway for the positioning of a clothing engaged belt of the user therein.

Further objectives of the ostomy collection support system herein will be brought out in the following parts of the specification wherein the summary and detailed description of the invention are for the purpose of fully disclosing the invention without placing limitations thereon.

### SUMMARY OF THE INVENTION

The present invention provides a device and method solving the shortcomings and dilemma noted in the prior art of ostomy support and collection belts and the like. The ostomy support system herein described and disclosed provides for a cavity which forms a surrounding protective support preventing compression of collection pouches and the like, employed for waste collection. The device features a housing which has a curved or arched protective member portion having ends engaged at both ends to a base member. In-between the base member and curved protective member portion is formed a cavity.

This formed cavity provides a protective location for positioning of a collection pouch. Positioned therein the pouch is provided support and also provided protection from impacts against the pouch during use. The arched member can also include one or a plurality of projections extending from the exterior surface of the arched member, reinforcing the strength of the member similar to the construction of an I-beam.

The housing is adapted for engagement at opposing ends to an elastic or other belt. This belt so engaged, when also operatively positioned to surround a portion of the body of the user such as the waist of the user, will serve to hold the housing in position against the body of the user in a compressive frictional engagement such as against the abdomen area.

Additionally provided on an exterior facing surface of the elongated member, which is on a side thereof opposite that of the cavity for the pouch, is a plurality of projections which not only significantly increase the bend resistance of the elongated member but also form a belt pathway. This belt pathway is configured to operatively hold a belt engaged with the user's pants or overflying clothing, within the belt pathway. This belt pathway is formed adjacent to at least one lower projection or preferably in-between a pair of opposing projections extending from the exterior surface of the elongated member. These projections, either singularly or in a pair, additionally serve to form a significant reinforcement to any bending of the elongated member. So positioned, the projections prevent the elongated member from bending toward the body of the user and the cavity, thereby protecting the underlying pouch from impacts. While an elongated member is shown for ease of disclosure the member could also be of a rectangular or other configuration where both ends extend substantially normal to the underlaying attachment or in other configurations to form the member covering the cavity. However, as a curved or arch shape is stronger, such is preferred.

In addition, while the device herein is being described, for convenience, as being waist or abdomen-positioned, it can also be mounted on a leg or ankle. Further, while it is described as being employable for an ostomy pouch, it can also be used for a pouch to collect urine or other fluids, whether mounted on the abdomen or the leg or ankle.

With respect to the above description, before explaining at least one preferred embodiment of the herein disclosed ostomy pouch support and protective device in detail, it is to be understood that the invention is not limited in its application to the details of construction and to the arrangement in the following description or illustrated in the drawings. The disclosed ostomy pouch support and protective system herein described is capable of other embodiments and of being practiced and carried out in various ways which will become obvious to those skilled in the art upon reading this disclosure. Also, it is to be understood that the phraseology and terminology employed herein are for the purpose of description and should not be regarded as limiting.

As such, those skilled in the art will appreciate that the conception upon which this disclosure is based, may readily be utilized as a basis for the designing of other ostomy collection pouch supports and protectors and for carrying out the several purposes of the present disclosed device. It is important, therefore, that the claims herein be regarded as including such equivalent construction and methodology, insofar as they do not depart from the spirit and scope of the present invention.

### BRIEF DESCRIPTION OF DRAWING FIGURES

The accompanying drawings, which are incorporated herein and form a part of the specification, illustrate some, but not the only or exclusive examples of embodiments and/or features of the ostomy collection pouch support and protection system. It is intended that the embodiments and figures disclosed herein are to be considered illustrative rather than limiting.

In the drawings:

FIG. 1 shows an overhead perspective view of the housing of the device herein having a curved protective member engaged at opposing ends with a base which is preferably flexible and showing ends adapted for engagement with an elastic belt which also may be provided engaged with a base member.

FIG. 2 is an overhead plan view of the housing of the device of FIG. 1, showing an ostomy pouch operatively positioned within the cavity protected from impact between the central portion of the elongated member in-between opposing ends which is spaced apart from the base member of the housing.

FIG. 3 depicts another perspective view of the device of FIG. 1.

FIG. 4. is an overhead side view of the housing of the device herein, showing the protective cavity defined by the second surface of the base member and an interior surface of a central portion of the elongated member located between opposing ends thereof which are connected to the base member of the housing, and showing a belt engaged to one side of the housing.

FIG. 5 is an opposite side view of the device from that of FIG. 4.

### DETAILED DESCRIPTION OF THE PREFERRED EMBODIMENTS OF THE INVENTION

In this description, any directional prepositions if employed, such as up, upwardly, down, downwardly, front, back, first, second, top, upper, bottom, lower, left, right and other such terms referring to the device or depictions as such may be oriented, are describing it such as it appears in the drawings and are used for convenience only. Such terms of direction and location are not intended to be limiting or to imply that the device herein has to be used or positioned in any particular orientation.

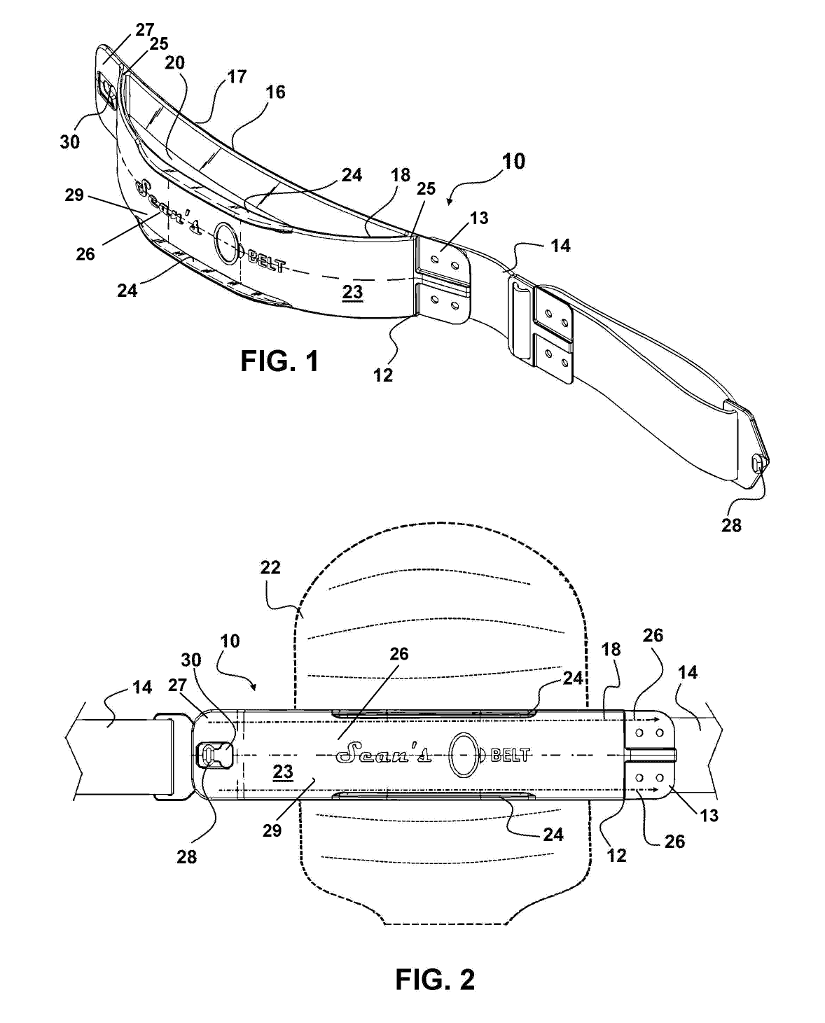

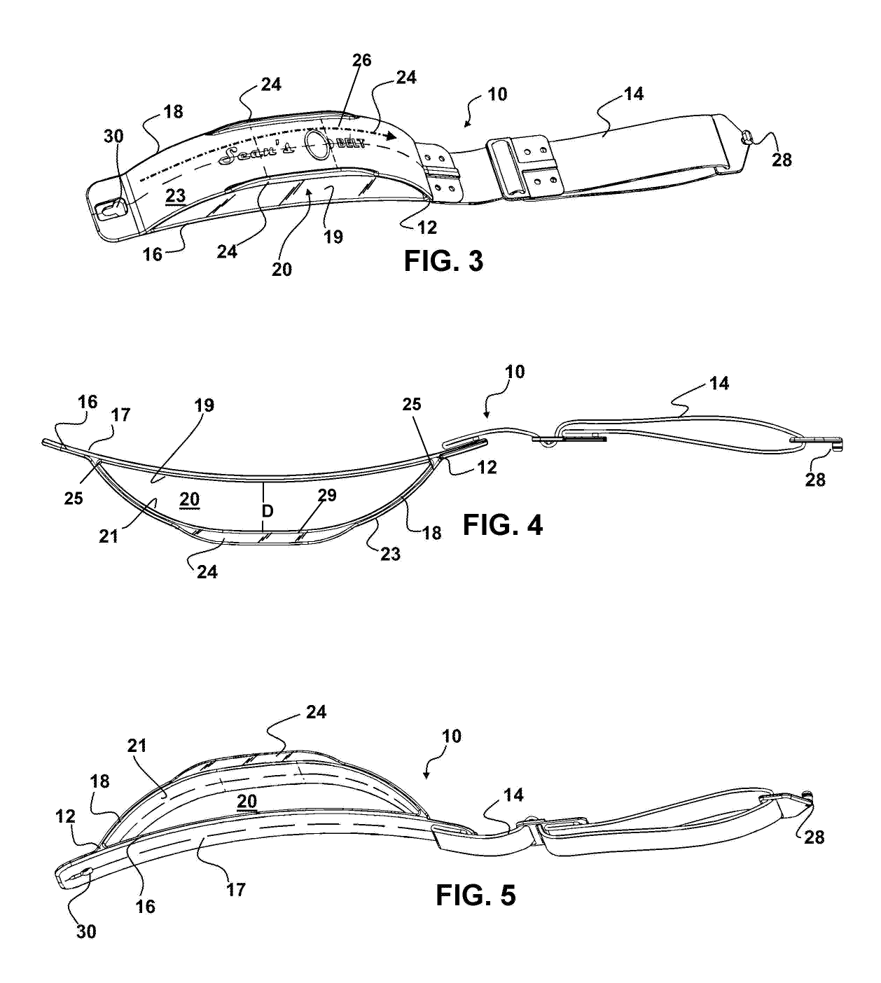

Now referring to drawing of FIGS. 1-5, where similar components are identified with like numeral references, there is seen in FIG. 1 an overhead perspective view of the device 10 showing a housing 12 having a base member 16 engaged to or adapted at a first end 13 for engagement with a belt 14. The belt 14 so engaged will hold the housing against a position on the body of a user when wrapped around a portion of the body of the user. The housing 12, including the base member 16 and elongated member 18, is best formed of a lightweight yet strong material such as a plastic or polymeric material or other lightweight metal or carbon fiber or fiberglass or the like but could also be of a flexible metal.

As shown in FIGS. 1-5, the housing 12 has a base member 16 having a first side surface 17 adapted to contact the body of the user, such as the skin of the user. The base member 16 has a second side surface 19 opposite the first side surface 17 which is engaged with opposing ends 25 of an elongated member 18. This elongated member 18 engaged at opposing ends 25 thereof to the base member 16 which as shown is preferably curved or arched in shape. This curved or arched shape is preferred because such increases the resistance to deformation of the elongated member 18 while concurrently projecting less from the base member 16, to make the device 10 more discreet. In the curved or arched configuration increases the protection against compression and deformation into the underlying cavity 20 which is adapted for positioning of a collection pouch 22 (FIG. 2) therein. However, the cavity 20 may be formed in-between the central portion 29 of the elongated member 18 which is located in-between the opposing ends 25 thereof, and the second side surface 19 of the base member 16 in other shapes as would occur to those skilled in the art. Such might be rectangular or other curved shapes and provide compressive protection but in a less compact and slightly less resistive elongated member 18 of the device 10.

The base member 16 is flexible and may be initially planar so long as it will bend to conform to the body of the user. Preferably the base member 16 is formed slightly curved such as shown in FIG. 4, to help it better conform to the curve of the abdomen of a user wearing the device 10 and make it more comfortable to wear by reducing increased force on the belt 14 a planar base member 16 forced to a curve causes. Additionally, the base member 16 is preferably flexible to allow it to flex slightly when worn to conform the first side surface 17 to the shape of the body of the user, and the polymeric or other flexible material chosen for the base member 16, should take such into consideration.

As can be seen in FIGS. 1-5, formed in between the second side surface 19 of the base member 16 and an interior surface 21 of the central portion 29 of the elongated member 18 in-between the opposing ends 25 engaged to the base member 16 is formed a cavity 20. This cavity 20 is adapted to surround and protect a collection pouch 22 operatively positioned therein during use of the device 10. Currently, a preferred distance of a widest diameter D (FIG. 4) of the cavity 22, located at a central section thereof in-between the tapering distance at opposing ends 25 of the elongated member 18, which provides both support and protection to a pouch engaged therein, is between inch to 2.5 inches. This provides for a surrounding protection of a pouch 22 and maintains the distance of the elongated member 18 from the base member 16 to make the device discreet under clothing.

As can be seen in all depictions of the device 10 in FIGS. 1-5, the elongated member 18 being engaged at opposing ends 25 to the base member 16, expending in a bend or arch or curve at a spaced distance from the second side surface 19 of the base member 16 is well positioned to prevent or limit impacts to a pouch 22 located within the cavity 20 for the pouch 22 covered over by the elongated member 18. It is the curved shape of the elongated member 18 which best resists bending or flexing in a direction toward the base member 16, and thereby forms a barrier to impacts to the surrounded pouch 22.

Such impacts can occur for example when a user accidently walks into a pole or door, or where an object such as a ball is thrown toward the user and not caught before impacting the elongated member 18, or for example when sitting in a vehicle and having a seat belt contacting the front of the abdomen and chest of the user. While the curved shape of the elongated member 18 forming an arched wall for the cavity 20 served well to prevent such impacts or at least mitigate their force, it was found the positioning of at least one, and preferably a plurality of elongated projections 24 extending from an exterior surface 23 of the elongated member 18, across all or a portion of the central portion 29 thereof, formed stiffeners which increase the resistance to deformation of the central portion 29 of the elongated member 18 toward the cavity 20. Further, with two elongated projections 24 running spaced and substantially parallel on or adjacent opposing sides of some or all of the central portion 29 elongated member 18, a belt pathway 26 (FIGS. 1-2) is formed along the exterior surface 23 of the elongated member 18 in-between the two elongated projections 24.

The pair of projections 24 also formed a pair of stiffeners which significantly enhanced the resistance to bending of the central portion 29 of the elongated member 18 toward the cavity 20, and therefor the protective resistance to impacts to the pouch 22. As such, the positioning of elongated projections 24 running along or adjacent opposing edges of the central portion 29 of the elongated member 18, with a space or gap therebetween defining a belt pathway 26, and having first ends engaged to the exterior surface 23 of the elongated member 18 and extending from the exterior surface thereof a distance to distal ends of the projections 24, is particularly preferred.

This belt pathway 26 (FIGS. 1-2) is preferred on the exterior surface of the elongated member 18 in-between the two opposing projections 24 in the favored modes of the device 10. It has been found in experimentation that this belt pathway 26 allows the user to position a belt such as that for pants, and the engaged portion of the pants, into a compressive positioning within the belt pathway 26 and in-between the two projections 24. This positioning of the belt and clothing into a compressed positioning aided in maintaining the housing 12 in a proper position on the abdomen of the user, as well as providing an enhanced disguise of the small mound which the housing 12 forms under the clothing thereby making the device 10 much more discreet which is particularly preferred by some users.

As can be seen throughout drawings 1-5, the belt 14 is engageable to or engaged at a first end 13 of the housing 12 and extends to a second end of the belt 14 where a cooperative connector 28 is positioned. This cooperative connector 28 is configured to removably engage with a mating connection 30, which is located at the second end 27 of the housing 12, opposite the first end 13 to which the belt is engaged. As shown, the mating connection 30 is an opening, such as a keyhole shaped opening, configured to removably engage with the cooperative connector 28 positioned therein as in FIG. 2.

While all of the fundamental characteristics and features of the ostomy pouch support and protective device herein have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that upon reading this disclosure and becoming aware of the disclosed novel and useful ostomy pouch support and protective system herein disclosed, that various substitutions, modifications, and variations may occur to and be made by those skilled in the art, without departing from the spirit or scope of the invention. Consequently, all such modifications and variations and substitutions, as would occur to those skilled in the art are considered included within the scope of the invention as shown and disclosed herein as defined by the following claims.

What is claimed is:

1. An ostomy support apparatus, comprising:
a housing extending between a first end and a second end;
said housing having a base member positioned between said first end and said second end thereof;
said base member having a first side surface and having a second side surface opposite said first side surface;
an elongated member having two ends and a central portion running therebetween, said two ends being connected with said base member;
said elongated member having an interior surface running between said two ends and positioned opposite said second side surface of said base member;
a cavity formed between said second side surface of said base member said interior surface of said elongated member;
said cavity adapted for positioning of a pouch therein;
said first end of said housing engageable with a first end of a belt and said second end of said housing engageable with a second end of said belt, whereby said housing positionable to an engaged position upon the body and held in said engaged position by said belt engaged to said first end and second end of said housing and around a portion of said body of said user.

2. The ostomy support apparatus of claim 1, additionally comprising:
said elongated member having an exterior surface opposite said interior surface;
at least one elongated ridge engaged at a first end to said exterior surface of said elongated member along said central portion thereof, and extending to a distal end of said elongated ridge; and
said elongated ridge forming a stiffener upon said central portion of said elongated member to resist a deflection thereof in a direction toward said cavity.

3. The ostomy support apparatus of claim 1, additionally comprising:
said elongated member having an exterior surface opposite said interior surface;
a pair of elongated ridges engaged at respective first ends to said exterior surface of said elongated member along a portion of said central portion thereof, each of said elongated ridges extending to a distal end; and
each of said elongated ridges forming a respective stiffener upon said central portion of said elongated member to resist a deflection thereof in a direction toward said cavity.

4. The ostomy support apparatus of claim 3, additionally comprising:
said exterior surface of said elongated member in-between said pair of elongated ridges defining a belt pathway; and
said belt pathway configured for positioning of a clothing belt engaged with clothing overlaying said housing, into a compressed positioning within said belt pathway.

5. The ostomy support apparatus of claim 1, additionally comprising:
said elongated member having an arched shape in-between said two ends thereof.

6. The ostomy support apparatus of claim 2, additionally comprising:
said elongated member having an arched shape in-between said two ends thereof.

7. The ostomy support apparatus of claim 3, additionally comprising:
said elongated member having an arched shape in-between said two ends thereof.

8. The ostomy support apparatus of claim 4, additionally comprising:
said elongated member having an arched shape in-between said two ends thereof.

9. The ostomy support apparatus of claim 1, additionally comprising:
said base member being curved in shape.

10. The ostomy support apparatus of claim 2, additionally comprising:
said base member being curved in shape.

11. The ostomy support apparatus of claim 3, additionally comprising:
said base member being curved in shape.

12. The ostomy support apparatus of claim 4, additionally comprising:
said base member being curved in shape.

13. The ostomy support apparatus of claim 5, additionally comprising:
said base member being curved in shape.

14. The ostomy support apparatus of claim 6, additionally comprising:
said base member being curved in shape.

15. The ostomy support apparatus of claim 7, additionally comprising:
said base member being curved in shape.

16. The ostomy support apparatus of claim 8, additionally comprising:
said base member being curved in shape.
